(19) United States
(12) Patent Application Publication
(10) Pub. No.: US 2014/0358157 A1
(43) Pub. Date: Dec. 4, 2014

(54) APPARATUSES AND SYSTEMS FOR KNOTLESS ABDOMINAL SACRAL COLPOPEXY

(71) Applicant: NATHAN GUERETTE, MIDLOTHIAN, VA (US)

(72) Inventor: NATHAN GUERETTE, MIDLOTHIAN, VA (US)

(21) Appl. No.: 14/294,920

(22) Filed: Jun. 3, 2014

Related U.S. Application Data
(60) Provisional application No. 61/830,387, filed on Jun. 3, 2013.

(51) Int. Cl. A61B 17/42

(52) U.S. Cl. CPC A61B 17/42
USPC 606/119

(57) ABSTRACT

The present invention provides for an apparatus and system for knotless abdominal sacral colpopexy. The apparatus and system includes a vaginal manipulator comprising: a central body connected to a tapered handle with a central bore hole configured throughout at least the central body and/or the tapered handle suitable to receive a uterine stabilizer. The apparatus and system further includes a screw lock in the central body, a uterine stabilizer configured to fit within the central bore hole and a vaginal cuff adapter.

### RELATED APPLICATIONS

The present invention relates to and claims priority Provisional Patent Application Ser. No. 61/830,387 entitled “APPARATUSES AND SYSTEMS FOR KNOTLESS ABDOMINAL SACRAL COLPOPEXY” filed Jun. 3, 2013.

### FIELD OF THE INVENTION

The invention relates generally to apparatuses, kits, and systems for performing abdominal sacral colpopexy for the robotic, laparoscopic, or open treatment of complex, multi-compartment female pelvic organ prolapse. In particular a vaginal manipulator and knotless sacral attachment device are presented. Kits comprising the vaginal manipulator and other devices are also presented.

### BACKGROUND OF THE INVENTION

Pelvic organ prolapse (POP) is a common disorder and surgical demand for its correction is growing rapidly with the aging population and increasing demands of active, older women. Abdominal sacral colpopexy (ASC) is considered the “gold standard” surgical treatment for POP but its use has been restricted due to its complexity, recovery time, and invasiveness.

Robotic and laparoscopic modifications of ASC have altered the procedure to a minimally invasive surgery with rapid recovery. The increased availability of robotic surgical equipment has lead to rapid growth of ASC as a viable option for the surgical treatment of POP. However, the lack of availability of specific equipment, duration of the surgery, and lack of standardized training remain barriers to its use.

### BRIEF SUMMARY OF THE INVENTION

The present invention provides a kit comprising a vaginal manipulator and a knotless sacral attachment device for use in performing a sacral colpopexy. Additionally, due to the specific design of the instruments it enables the procedure to be performed in a faster, stronger, and safer manner. Further, as it is performed with a standard technique, it facilitates focused training efforts to enable broader use of ASC.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIGS. 1A-1D illustrates views of one of the present embodiments of a vaginal manipulator for knotless abdominal sacral colpopexy.

FIGS. 2A-2B illustrate views of one of the present embodiments of a vaginal manipulator for knotless abdominal sacral colpopexy.

FIGS. 3A-3B illustrate one of the present embodiments of the vaginal cuff adapter.

FIG. 4 illustrates one of the present embodiments of a quill mesh attachment.

FIGS. 5A-5B illustrate views of one of the present embodiments of a knotless sacral attachment device.

### DETAILED DESCRIPTION

Embodiments of a kit 10 for ASC procedures are discussed below.

In one embodiment, kit comprises: a vaginal manipulator 100, a mesh 500, a Uterine/Cervical Stabilizer rod 150, a vaginal cuff adapter 104, a plurality of quill SRS sutures 300, and at least one knotless sacral attachment (KSA) device 400. Vaginal manipulator 100 is configured to aid in the performance of an ASC, whether or not a hysterectomy is performed concomitantly. Vaginal manipulator 100 is configured for simple and rapid placement and will allow the surgeon to perform a supracervical hysterectomy (SCH) and ASC without having to change instruments or remove instruments during the surgery. Vaginal manipulator 100 is configured to distend the vagina for superior dissection and graft attachment. In addition, vaginal manipulator 100 comprises stabilizer rod 150, which rod is configured to fix the cervix to aid in accuracy and speed of dissection and graft placement.

The vaginal manipulator 100, in one embodiment, consists of a cylindrical solid plastic barrel with a central bore hole, such as in one embodiment having a bore hole of approximately 0.25 cm, to allow placement of the uterine manipulator 150 or vaginal cuff adapter 200, as described in further detail below. The apical end on the cylinder has a concave defect “cup” 104 with a 2×3 cm dimension, in one embodiment, to allow fixation and stabilization of the cervix during ASC. The overall dimensions of the central cylinder are 12×4 cm in one embodiment. The central cylinder (body) is attached to a handle, which may be tapered, (also 12 cm in length in one embodiment) to aid in manipulation.

In one embodiment, the tapered handle includes the bore hole through the entire length and a locking mechanism 190 to fix either the uterine/cervical stabilizer or vaginal cuff adapter in place. In one embodiment, the locking mechanism may be a butterfly screw, or any other suitable locking mechanism.

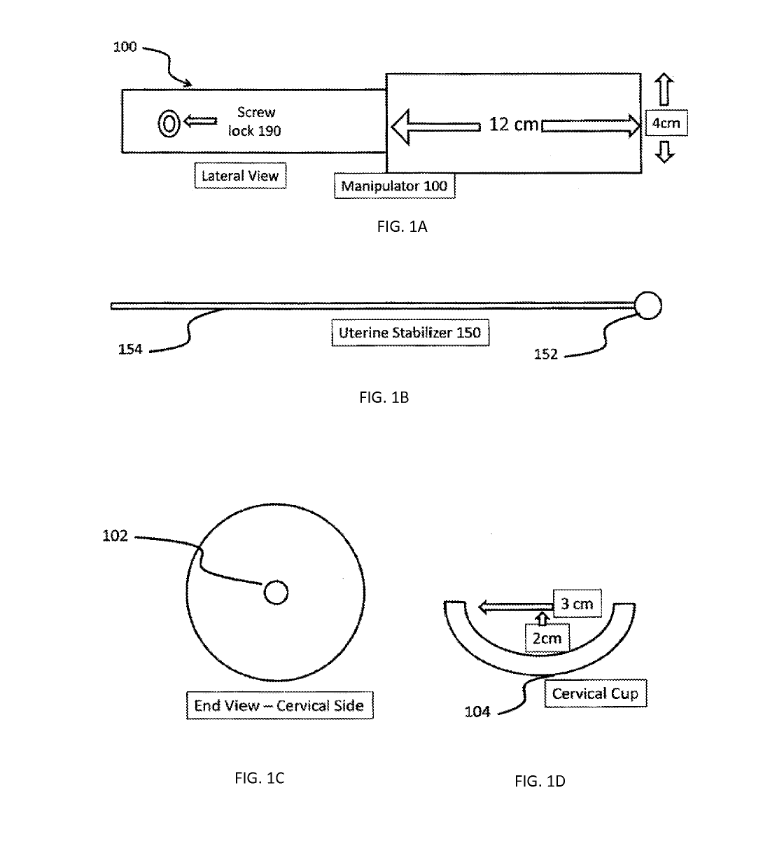

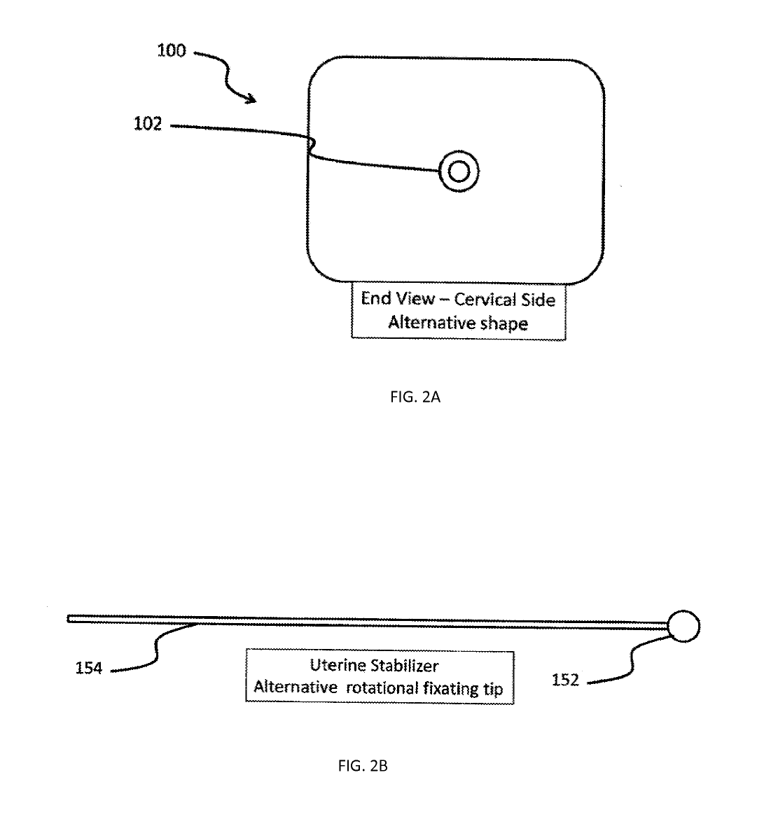

In one embodiment presented the central cylinder is completely circular in design (FIG. 1A), in another, it is a rounded rectangle on cross sectional view (FIG. 2A).

In one embodiment, the Uterine/Cervical Stabilizer Rod 150 (UCSR, FIG. 1B) is also constructed of plastic and consists, in one embodiment, of a 40×0.25 cm shaft 154 and a rounded 0.5 cm tip 152. The rod 150 is designed to be placed into the endometrial cavity after cervical dilatation and then have the vaginal manipulator placed and locked (using the bore hole over the shaft and locking mechanism) to aid in manipulation of the uterus during supracervical hysterectomy (SCH). The above-noted dimensions represent one exemplary embodiment and such dimensions are not limiting in nature.

Once the hysterectomy is complete the UCSR may be withdrawn into the cervix for stabilization during ASC without having to remove the vaginal manipulator. In one embodiment the end is a simple rounded, spherical tip, having in one example a dimension of 0.5 cm, in another the tip may rotate with deployment of fixating barbs that are controlled from the external end to fix the tip to the uterus or cervix for improved control. Rotation of the end in the opposite direction will release the barbs and withdraw them into the tip.

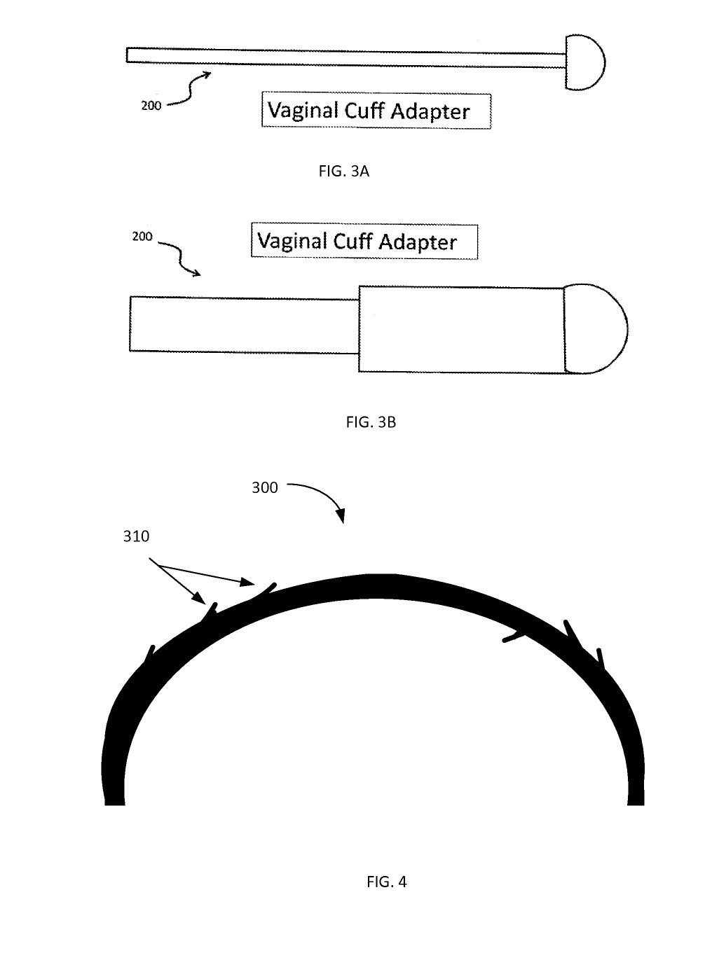

The vaginal cuff adapter 200 (FIGS. 3A, B) is also constructed of plastic and, in one embodiment, has a 40×0.25 cm shaft with a 4×2 cm rounded end at one side. The above-noted dimensions represent one exemplary embodiment and such dimensions are not limiting in nature. It is designed to fit and lock into the apical end of the vaginal manipulator to assist in the performance of ASC in a patient with a previous complete hysterectomy. The wide rounded tip aids in establishing an adequate platform for dissection of the anterior and posterior vagina from the bladder and rectum and also aids in vaginal distention for easier and more accurate placement on the vaginal portion of the ASC mesh. In addition, the seamless attachment to the vaginal manipulator allows clear distention and identification of the entire vagina to further ease the procedure.

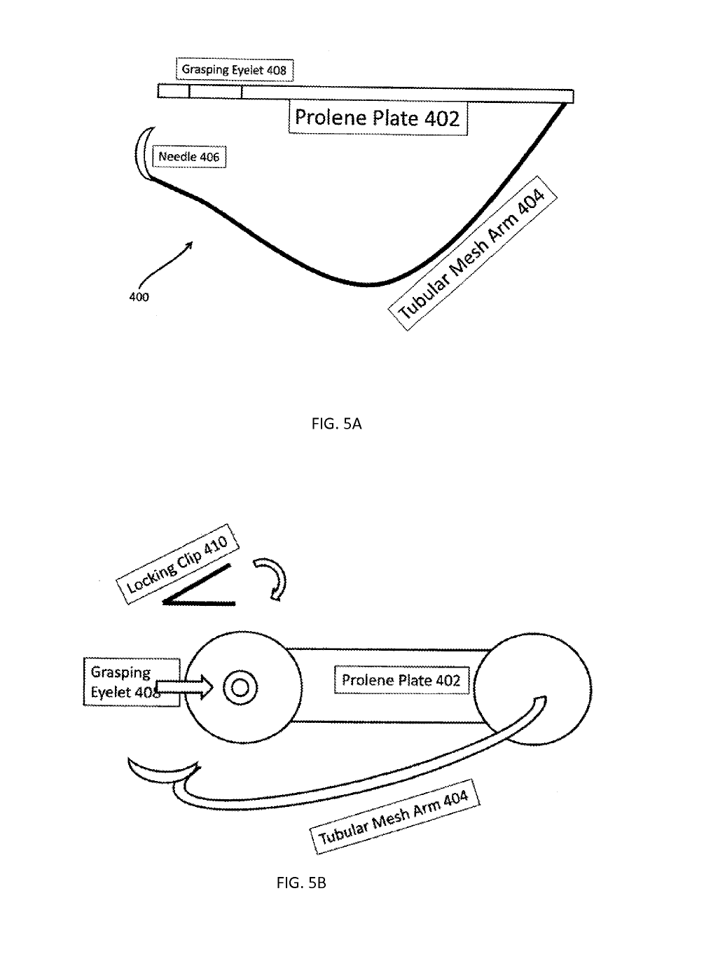

FIGS. 5A and 5B illustrate lateral and end views of a knotless sacral attachment (KSA) device 400. KSA device 400 includes a prolene plate 402, a tubular mesh arm 404 coupled to prolene plate 402, and a needle 406 coupled to tubular mesh arm 404. Prolene plate 402 also comprises a grasping eyelet 408 configured to receive a locking clip 410. In certain embodiments, prolene plate 402 is 2 cm long, but it is recognized that any suitable distance may be utilized.

KSA device 400 is configured for fixation of the apical portion of the ASC “Y” mesh to the anterior longitudinal ligament of the sacrum by passing needle 406 and tubular mesh 404 through the ligament and then through the eye 408 then tensioning appropriately and locking the arm in place with the locking clip 410, thus locking the plate and the apical portion of the “Y” mesh to the sacrum without requiring a knot. Furthermore, the Knotless Sacral Attachment component would increase the strength of the sacral attachment by providing a broad plate rather than narrow suture attachment and avoid the need to tie knots but maintain a standard needle type pass through the anterior longitudinal ligament rather than using tacking devices that violate the periosteum of the sacrum.

FIG. 4 illustrates one embodiment of quill SRS suture 300. Suture 300 comprises a plurality of quills 310 configured to attach the anterior and posterior vaginal mesh leaves 500 to the anterior, posterior and apical vagina/cervix in a knotless fashion as well as recover the mesh 500 with the retropertitoneum.

The restorelle “Y” mesh 500 is a type of macroporous monofilament polypropylene mesh designed specifically for the performance of ASC. In one embodiment, the mesh may be manufactured by Mpathy Medical/Colplast Corporation. The mesh possesses a linear box weave to aid in the passage of running suture material. It is comprised of an apical, anterior, and posterior leaf (24×4 cm or 27×4 cm total dimension) that may be fashioned to conform to individual anatomy. The mesh 500 would be secured to the anterior and posterior vagina as well as the apex or cervix with running, non-locking Quill SRS suture 300 using the vaginal manipulator 100 as a superior platform for vaginal distention and stabilization.

In addition to the kit described above, the utilization of the kit provides for the performance of knotless abdominal sacral colpopexy.

In a first step, at the initiation of the version of the procedure that would include a SCH as well as an ASC the cervix would be dilated and the UCSR 150 would be placed into the full depth of the endometrial cavity. The vaginal manipulator 100, using the bore hole, is passed over the shaft of the UCSR to the full vaginal length and the UCSR is secured to the vaginal manipulator using the screw-lock 190 thus achieving superior control of the uterus and vagina for hysterectomy, dissection and graft placement.

In a second step, the SCH is performed in a standard fashion and once the uterine fundus was ready to be removed the UCSR 150 would then be mobilized by loosening the screw 190 and then withdrawn until the spherical tip was in the cervix to maintain complete control of the remaining tissue. The uterine fundus is then be removed.

In a third step, once the uterine fundus is removed the combination of the vaginal manipulator 100 and the UCSR 150 act as a superior platform for angulation and distention of the vagina and cervix while the bladder is dissected off the anterior vagina and the rectum off the posterior vagina.

In a fourth step, the retroperitoneal dissection and exposure of the anterior longitudinal ligament of the sacral promontory is then performed in a standard fashion.

In a fifth step, once the dissection is complete, the Restorelle “Y” mesh 500 would be appropriately measured and brought into the peritoneal cavity. The anterior and posterior leaves of the mesh would then be attached to the anterior and posterior vagina with the Quill SRS suture 300.

In a sixth step, once the mesh is attached, the vaginal manipulator 100 is used to provide appropriate vaginal support and the KSA 400 would be introduced. The tubular arm of the KSA 404, 406 would be passed through the apical portion of the mesh 500 at an appropriate location and also though the anterior longitudinal ligament in a transverse direction. The arm is further passed through the locking eyelet 408 on the opposite side of the KSA plate, tensioned to lay flat on the sacrum, and locked in place with the locking clip 410. This can be repeated with multiple KSA devices.

A seventh step, the can be completely retroperionealized using a Quill SRS suture to prevent contact with intrapertioneal contents and internal incarcerated hernias.

Therein, the remainder of the procedure is completed as indicated.

### Procedure Variation (Prior Complete Hysterectomy)

In the case of a patient with a prior complete hysterectomy the vaginal manipulator 100 can be attached to the vaginal cuff adapter 200 using the screw lock 190 at the initiation of the surgery. The combined apparatus would then be placed vaginally for adequate vaginal distension and stabilization. The remainder of the procedure would be performed according to steps 3•8 as listed above.

In the specification and in the claims, the terms “including” and “comprising” are open-ended terms and should be interpreted to mean “including, but not limited to . . . .” These terms encompass the more restrictive terms “consisting essentially of’ and “consisting of.”

It must be noted that as used herein and in the appended claims, the singular forms “a”, “an”, and the include plural reference unless the context clearly dictates otherwise. As well, the terms “a” (or “an”), one or more and at least one can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Although the present invention has been described with reference to preferred embodiments, persons skilled in the art will recognize that changes may be made in form and detail without departing from the spirit and scope of the invention.

Such changes and modifications may include dimensions of the elements noted herein, wherein all provided dimensions noted above are exemplary in nature. The providing of dimensions as recited herein are not limiting or expressly or inherently excluding other dimensions, for example the central cylinder is noted above as having an exemplary dimension of 12×4 cm, whereby it is within the scope of the such that the dimensions may be any suitable range consistent with the operations and functional utilizations described herein, as recognized by one skilled in the art.

All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A kit comprising:
a vaginal manipulator comprising: a central body connected to a tapered
handle with a central bore hole configured throughout at least the central body and/or the tapered handle suitable to receive a uterine stabilizer;
a screw lock in the central body;
a uterine stabilizer configured to fit within the central bore hole; and a vaginal cuff adapter.

2. A sacral attachment device comprising:
a prolene plate having a
proximal end and a distal end, a grasping eyelet at the proximal end, a tubular mesh arm coupled to the prolene plate at the distal end, and a needle coupled to the tubular mesh arm designed to fix a vaginal mesh to the anterior longitudinal ligament of the sacrum in a knotless manner.
